(19) United States
(12) Patent Application Publication
(10) Pub. No.: US 2022/0266210 A1
(43) Pub. Date: Aug. 25, 2022

(54) PRODUCTION METHOD FOR LIPID PARTICLES IN LIQUID AND METHOD FOR CULTURING MICROORGANISMS

(71) Applicant: KANEKA CORPORATION, Osaka-shi (JP)

(72) Inventors: Takafumi IGARI, Hyogo (JP); Shihomi NISHIMORI, Hyogo (JP); Masaru HIRANO, Hyogo (JP); Hiroaki ICHIRIKI, Hyogo (JP); Akihisa KANDA, Hyogo (JP)

(73) Assignee: KANEKA CORPORATION, Osaka-shi (JP)

(21) Appl. No.: 17/742,035

(22) Filed: May 11, 2022

Related U.S. Application Data
(63) Continuation of application No. PCT/JP2020/040062, filed on Oct. 26, 2020.

(30) Foreign Application Priority Data
Nov. 25, 2019 (JP) 2019-212566

(51) Int. Cl. B01J 2/06; C12N 1/20

(52) U.S. Cl. CPC B01J 2/06; C12N 1/20; C12N 2330/00

(57) ABSTRACT

A method for producing lipid particles, including: injecting molten lipids directly into a liquid at a temperature lower than a melting point of the lipids through a liquid supply port of a two-fluid nozzle while injecting a gas directly into the liquid through a gas supply port of the two-fluid nozzle, so that the molten lipids are dispersed and atomized into particles in the liquid due to the gas and the particles are solidified to form lipid particles. The lipids have a water solubility of 10 g/L or less at 25° C. and are solid at 25° C. The two-fluid nozzle is heated to a temperature at least 10° C. higher than the melting point of the lipids. A ratio D50/Nd of a volume median diameter D50 of the lipid particles to an orifice diameter Nd of the liquid supply port of the two-fluid nozzle is 0.0017 or more and 0.17 or less.

### TECHNICAL FIELD

The present invention relates to a method for producing fine particles of lipids in a liquid. Specifically, the present invention relates to a method for producing lipid particles, in which lipids that are assimilable by microorganisms and less soluble in water are atomized in a liquid, and also relates to a method for culturing microorganisms.

### BACKGROUND ART

People are becoming more conscious of environmental issues, food issues, health, and safety. There is also a growing awareness of nature. Under these circumstances, the significance and importance of the production of microorganisms and the microbial production of substances (fermentation production, bioconversion, etc.) are increasingly prominent.

The production of microorganisms and the microbial production of substances require carbon sources that can be satisfactorily assimilated by microorganisms (i.e., carbon sources for culture, fermentation, or the like). Typical examples of the carbon sources include carbohydrates and lipids (such as animal and vegetable fats and oils including fatty acids).

However, some lipids have a higher melting point than the culture temperature of microorganisms and a very low solubility in water. Such lipids coagulate in a culture solution, have low assimilability by microorganisms, and thus cannot be used appropriately. For example, the solubility of high melting point fatty acids such as lauric acid, myristic acid, palmitic acid, and stearic acid is at most 0.1 g/L or less at a water temperature of 25° C., even for the highest water-soluble lauric acid.

Several production examples have been reported in which fatty acids and their salts are used as carbon sources having a higher melting point than the culture temperature of microorganisms. One example is that lauric acid is used alone as a carbon source to culture Aeromonas hydrophila (Non-Patent Document 1). In this example, the amount of dried bacterial cells remains at about 8 g/L. Non-Patent Document 1 shows that the lauric acid is solid at the culture temperature, which increases the difficulty in culture.

Moreover, an example has been reported in which a fatty acid is prepared in the form of an oil-in-water emulsion to have a large specific surface area, and this emulsion is supplied to a culture solution as a carbon source, so that the growth of bacterial cells can be improved (Patent Document 1). Another example has also been reported in which fat or oil is heated to a temperature higher than their melting points, and then the heated fat or oil is added to a culture medium in a dispersed state and used for the production of bioproducts by microorganisms (Patent Document 2).

### PRIOR ART DOCUMENTS

### Patent Documents

Patent Document 1: WO 2013/057962 A1
Patent Document 2: WO 2013/016690 A1

### Non-Patent Documents

Non-Patent Document 1: Lee S. et al., Biotechnol. Bioeng., 67:240-244(2000)

### DISCLOSURE OF INVENTION

### Problem to be Solved by the Invention

However, the technology disclosed in Patent Document 1 involves the costs of an emulsifier, preparation equipment, and a reservoir. The storage period is limited when the emulsifier is a protein. Thus, there are still many problems with the application of this technology to industrial production in terms of economy. Moreover, a stirring speed in a culture tank needs to be controlled in order to control the diameter of oil drops. However, the stirring speed in the culture tank may affect the oxygen transfer coefficient kLa of the culture solution. Therefore, itis not easy to meet the ideal conditions for both the culture conditions and the diameter of droplets of fats and oils. An alternative method would be to produce lipid particles outside the culture tank and add the lipid particles to the culture solution. However, this method results in a very long manufacturing process and an increase in the cost of manufacturing facilities or the like.

Patent Document 2 specifically discloses only an example in which PFAD (palm fatty acid distillate) particles with a diameter as large as about 1 mm are dispersed by injecting the PFAD through an injector into a culture solution. In the technology of Patent Document 2, since the density of fatty acid is lower than that of water, the fatty acid is not easily dispersed in a liquid even if fatty acid droplets are added from above the culture solution by spraying. Further, the dispersion of the fatty acid becomes more difficult when the culture solution foams.

As described above, it has still been a challenge to achieve the industrially efficient production of microorganisms and microbial production of substances by appropriately using lipids that have a higher melting point than the culture temperature and a low solubility in water.

In view of the above problems, the present invention provides a method for producing lipid particles, in which lipids that have a low solubility in water at room temperature and are solid at room temperature are efficiently and finely dispersed in a liquid at a temperature lower than the melting point of the lipids, thereby providing lipid particles in the liquid. The present invention also provides a method for producing microorganisms.

### Means for Solving Problem

In one or more embodiments, the present invention relates to a method for producing lipid particles, in which molten lipids are solidified to form particles in a liquid. The lipids have a water solubility of 10 g/L or less at 25° C. and are solid at 25° C. The method includes injecting the molten lipids directly into a liquid at a temperature lower than a melting point of the lipids through a liquid supply port of a two-fluid nozzle while injecting a gas directly into the liquid through a gas supply port of the two-fluid nozzle, so that the molten lipids are dispersed and atomized into particles in the liquid due to the gas and the particles are solidified to form lipid particles. The two-fluid nozzle is heated to a temperature at least 10° C. higher than the melting point of the lipids. The ratio D50/Nd of a volume median diameter D50 of the lipid particles to an orifice diameter Nd of the liquid supply port of the two-fluid nozzle is 0.0017 or more and 0.17 or less.

In one or more embodiments, the present invention relates to a method for culturing microorganisms. The method includes preparing lipid particles in a culture solution by the above method for producing lipid particles, and culturing microorganisms in the culture solution containing the lipid particles.

### Effects of the Invention

According to the production method of the present invention, lipids that have a low solubility in water at room temperature (25° C.) and are solid at room temperature can be efficiently and finely dispersed in a liquid at a temperature lower than the melting point of the lipids, thereby providing lipid particles in the liquid.

According to the culture method of the present invention, lipids that have a low solubility in water at room temperature and are solid at room temperature are efficiently and finely dispersed and atomized into particles in a culture solution at a temperature lower than the melting point of the lipids. Thus, the lipid particles can be satisfactorily assimilated by microorganisms, and useful substances, e.g., microbial metabolites such as PHA can be produced with industrial efficiency.

### BRIEF DESCRIPTION OF DRAWINGS

FIG. 1 is a schematic diagram of a device for producing lipid particles used in one or more embodiments of the present invention.

FIG. 2 is a graph that plots data of Vg/Vf versus the ratio of the volume median diameter of PFAD particles to the orifice diameter of a liquid supply port in Examples 6 to 14.

### DESCRIPTION OF THE INVENTION

The present inventors conducted extensive studies to solve the above problems. As a result, the present inventors found the following method to produce lipid particles. Specifically, using a two-fluid nozzle that was heated to a temperature at least 10° C. higher than the melting point of lipids, molten lipids were injected directly into a liquid at a temperature lower than the melting point of the lipids through a liquid supply port of the two-fluid nozzle while a gas was injected directly into the liquid through a gas supply port of the two-fluid nozzle. Moreover, the ratio D50/Nd of the volume median diameter D50 of the target lipid particles to the orifice diameter Nd of the liquid supply port was set to 0.0017 or more and 0.17 or less. Thus, the molten lipids were finely dispersed in the liquid by contact with the gas and solidified to form lipid particles. In one or more embodiments of the present invention, the “liquid” refers to an aqueous solution or an aqueous dispersion containing water as the main solvent. In one or more embodiments of the present invention, “containing water as the main solvent” means that the solvent contains 90% by mass or more of water, preferably 95% by mass or more of water, and more preferably 100% by mass of water. In the present invention, the “molten lipids” means that the temperature of the lipids is equal to or higher than the melting point.

In one or more embodiments of the present invention, the lipids are not particularly limited, and have a water solubility of 10 g/L or less at 25° C. and are solid at 25° C. For example, the present invention may use any lipid that is assimilable by microorganisms. Examples of the lipids include fatty acids, hydrocarbons, sterols, and mixtures thereof. In particular, the lipids that can be utilized in the metabolic pathways of the target microorganisms are suitable.

Examples of the fatty acids include fatty acids, fatty acid salts, and fatty acid esters. The fatty acid salts may include, e.g., fatty acid sodium, fatty acid potassium, fatty acid calcium, and fatty acid magnesium. The fatty acid esters may include, e.g., fatty acid glycerol esters. The fatty acid glycerol esters may include, e.g., triglyceride, diglyceride, and monoglyceride.

Examples of the hydrocarbons include paraffin (paraffin wax), wax, polyethylene wax, petrolatum, and ceresin. Examples of the sterols include cholesterol and 24-methylenesterol.

The lipids are preferably animal and vegetable fats and oils, fatty acid glycerol esters derived from animal and vegetable fats and oils, or fatty acids derived from animal and vegetable fats and oils. In view of the impact on food issues, the lipids are more preferably non-edible fats and oils, fatty adds, or waste oil.

Examples of the animal and vegetable fats and oils include the following: beef tallow, lard; milk fat; fish oil; soybean oil; rapeseed oil; sunflower oil; olive oil; sesame oil; canola oil; peanut oil; tung oil; rice oil; cottonseed oil; rice oil; safflower oil; coconut oil; crude palm oil (CPO); crude palm kernel oil (CPKO); palm oil; palm kernel oil; shea butter; sal butter; illipe butter; cacao butter; jatropha oil; algae-derived fats and oils; and partially refined oil, fractionated oil, hardened oil, and interesterified oil of these fats and oils. Preferred examples of the animal and vegetable fats and oils include palm fractionated oil such as palm olein or palm double olein, which is a low-melting fraction of palm oil, and palm kernel fractionated oil such as palm kernel olein, which is a low-melting fraction of palm kernel oil. Examples of the fatty acids derived from animal and vegetable fats and oils include fatty acids, fatty acid salts, and fatty acid esters, which are constituents of the animal and vegetable fats and oils, as described above. These fats and oils may be used individually or in combination of two or more.

Specific examples of the lipids derived from palm oil include the following: crude palm oil; crude palm kernel oil; palm oil; palm kernel oil; palm olein; palm double olein; palm kernel olein; PFAD (palm fatty acid distillate); PKFAD (palm kernel fatty acid distillate); POME (palm oil mill effluent, which is a waste liquid discharged in the process of producing crude palm oil from oil palm fruit); EFB juice (empty fruit bunch juice, which is a by-product obtained in the process of producing empty fruit bunch pellets from the empty fruit bunch of oil palm); and partially refined oil, fractionated oil, hardened oil, and interesterified oil of these fats and oils.

The use of components that are produced as by-products in the process of refining the animal and vegetable fats and oils is preferred because it can avoid conflict with food. Examples of the by-products in the refining treatment include fatty acid salts produced in an alkaline deoxidation process, distillation residues produced in a distillation deoxidation process, and distillation residues produced in a deodorization process.

The fatty acid salts as by-products in the reining treatment may include, e.g., fatty acid sodium, fatty acid potassium, fatty acid calcium, and fatty acid magnesium. More specifically, the fatty acid salts may include, e.g., PFAD (palm fatty acid distillate), PKFAD (palm kernel fatty acid distillate), and fatty acid distillate of rapeseed oil.

The distillation residues as by-products in the refining treatment may contain monoglyceride, diglyceride, or the like in addition to the fatty acid as the main component.

In one or more embodiments of the present invention, the melting point of the lipids is preferably, e.g., 30° C. or more and 120° C. or less. The lipids with a melting point of 30° C. or more can easily be used by microorganisms as a carbon source. The melting point of the lipids is more preferably 35° C. or more, further preferably 38° C. or more, and particularly preferably 40° C. or more. The lipids with a melting point of 120° C. or less are less likely to coagulate in a nozzle, which can reduce the occurrence of a blockage in the flow path. The melting point of the lipids is preferably 100° C. or less, and more preferably 80° C. or less.

In one or more embodiments of the present invention, the lipids that have a water solubility of 10 g/L or less at 25° C. and are solid at 25° C. may be used optionally in combination with other components such as other carbon sources.

The two-fluid nozzle (also referred to as a pneumatic spray nozzle or an air spray nozzle) is not particularly limited. A known two-fluid nozzle may be appropriately used as long as the ratio D50/Nd of the volume median diameter D50 of the target lipid particles to the orifice diameter Nd of the liquid supply port is 0.0017 or more and 0.17 or less, and the liquid can be atomized. When the ratio D50/Nd is 0.0017 or more and 0.17 or less, fine lipid particles with a volume median diameter of, e.g., less than 1 mm can be suitably produced. The ratio D50/Nd is more preferably 0.005 or more and 0.14 or less, further preferably 0.01 or more and 0.10 or less, and particularly preferably 0.02 or more and 0.08 or less from the viewpoint of facilitating the formation of fine lipid particles.

The two-fluid nozzle is not particularly limited and is preferably an external mix nozzle in which a liquid and air are mixed outside the nozzle, e.g., in order to reduce clogging of the nozzle. With this configuration, the two-fluid nozzle injects (supplies) the molten lipids directly into a liquid at a temperature lower than the melting point of the lipids through the liquid supply port while injecting a gas directly into the liquid through the gas supply port. Thus, the molten lipids are dispersed and atomized into particles in the liquid due to the injected gas and the particles are solidified. Consequently, the lipid particles can easily be obtained. The two-fluid nozzle may be attached with the liquid injection hole and the gas injection hole located in the liquid at a position lower than the liquid level so that both the molten lipids and the gas can be injected directly into the liquid. The materials for the two-fluid nozzle are not particularly limited and may include, e.g., stainless steel, ceramic, and titanium because of their high stability in the liquid such as a culture solution.

The two-fluid nozzle is heated to a temperature at least 10° C. higher than the melting point of the lipids. This can reduce coagulation of the lipids in the nozzle. The heating temperature of the two-fluid nozzle is preferably at least 15° C. higher, and more preferably at least 20° C. higher than the melting point of the lipids. The heating temperature of the two-fluid nozzle is not particularly limited and is preferably 200° C. or less, more preferably 180° C. or less, and further preferably 160° C. or less, e.g., from the viewpoint of reducing the deterioration of the culture medium, the denaturation of secretions of microorganisms in the culture medium, the degeneration of cells of microorganisms, and then the adverse effect on growth. The heating method is not particularly limited.

The spray pattern of the liquid ejected from the nozzle is not particularly limited and is preferably a circular pattern because the spray flow rate is large and the particle size distribution tends to be uniform.

The ratio Vg/Vf of an injection linear velocity of the gas (Vg) to an injection linear velocity of the molten lipids (V) of the two-fluid nozzle is not particularly limited and is preferably 10 or more and 2000 or less, more preferably 12 or more and 1900 or less, further preferably 15 or more and 1800 or less, and particularly preferably 15 or more and 1200 or less, e.g., from the viewpoint of facilitating the atomization of the lipids in the liquid.

The lipids may be in a molten state when they are supplied directly into the liquid. The temperature of the molten lipids is not particularly limited and is preferably at least 5° C. higher, more preferably at least 10° C. higher, and further preferably at least 15° C. higher than the melting point of the lipids from the viewpoint of reducing the coagulation of the lipids in the nozzle and facilitating the atomization of the lipids in the liquid. The temperature of the molten lipids is not particularly limited and is preferably 150° C. or less, more preferably 120° C. or less, and further preferably 95° C. or less, e.g., from the viewpoint of reducing the deterioration of the culture medium, the denaturation of secretions of microorganisms in the culture medium, the degeneration of cells of microorganisms, and then the adverse effect on growth. The heating method is not particularly limited.

The gas is not particularly limited. For example, in aerobic culture, the gas is preferably air, oxygen, or a mixture of them because the gas ejected from the nozzle can be used to supply oxygen to microorganisms. In anaerobic culture, the gas is preferably nitrogen or a mixture of air and nitrogen because the dissolved oxygen concentration in the culture solution can be kept low. In hydrogen bacteria culture, the gas is preferably hydrogen or a mixture of air and hydrogen because the gas ejected from the nozzle can be used to supply hydrogen to microorganisms.

The temperature of the gas is not particularly limited and is preferably equal to or higher than the melting point of the lipids, more preferably at least 5° C. higher than the melting point of the lipids, and further preferably at least 10° C. higher than the melting point of the lipids, e.g., from the viewpoint of reducing the coagulation of the lipids in the nozzle and facilitating the atomization of the lipids in the liquid. The temperature of the gas is not particularly limited and is preferably 200° C. or less, more preferably 180° C. or less, and further preferably 160° C. or less, e.g., from the viewpoint of reducing the deterioration of the culture medium, the denaturation of secretions of microorganisms in the culture medium, the degeneration of cells of microorganisms, and then the adverse effect on growth. The heating method is not particularly limited.

The liquid is preferably stirred when the molten lipids and the gas are supplied directly into it, from the viewpoint of allowing the lipid particles to be widely dispersed in the liquid and to be more effectively made into fine particles. In this case, e.g., a stirrer with an impeller or the like may be used for stirring the liquid. The temperature of the liquid is lower than the melting point of the lipid particles and may be, e.g., 20° C. or more. When the liquid is a culture solution, the temperature of the culture solution may be, e.g., 25° C. or more and 37° C. or less.

The volume median diameter D50 of the lipid particles is preferably 150 μm or less, more preferably 80 μm or less, further preferably 60 μm or less, and particularly preferably 40 μm or less, e.g., from the viewpoint of facilitating the assimilation of the lipid particles by microorganisms as a carbon source. The volume median diameter D50 of the lipid particles is preferably 1 μm or more, more preferably 5 μm or more, and further preferably 10 μm or more, e.g., from the viewpoint of improving the atomization efficiency.

From the viewpoint of facilitating the dispersion of the lipid particles in the liquid and the assimilation of the lipid particles by microorganisms as a carbon source, the span in the particle size distribution of the lipid particles represented by the following formula (1) is preferably 0.5 or more and 3.0 or less, more preferably 0.8 or more and 2.8 or less, and further preferably 1.0 or more and 2.5 or less.

Span=(D90−D10)/D50 (1)

In one or more embodiments of the present invention, the particle size distribution of the lipid particles can be measured with a laser diffraction scattering method. The method may use, e.g., a particle size distribution measuring device “MT3300EX II” manufactured by Microtrac Inc.

It is preferable that the molten lipids and the gas are supplied directly into a culture solution and atomized from the viewpoint of facilitating the assimilation of the lipid particles by microorganisms as a carbon source. The molten lipids and the gas may be supplied into the culture solution collectively, continuously, or intermittently. Moreover, other carbon sources may be supplied directly into the culture solution at the same time as the molten lipids.

The lipid particles can be prepared in the culture solution by the above production method, and microorganisms can be cultured in the culture solution containing the lipid particles.

The microorganisms are not particularly limited and may be, e.g., microorganisms that are able to produce environmentally friendly biodegradable plastics having little adverse effect on the ecosystem. In particular, microorganisms that produce polyhydroxyalkanoates (also referred to as PHAs in the following) are preferred. PHAs are produced by using plant-derived natural organic acids or fats and oils as carbon sources, and serve as energy storage materials that are accumulated intracellularly.

The PHA is a general term for polymers containing 3-hydroxyalkanoic adds as monomer units. The 3-hydroxyalkanoic acids are not particularly limited and may include, e.g., 3-hydroxypropionate, 3-hydroxybutyrate, 3-hydroxyvalerate, 3-hydroxyhexanoate, 3-hydroxyheptanoate, and 3-hydroxyoctanoate. The PHA may be either a homopolymer containing one type of 3-hydroxyalkanoic acid as a monomer unit or a copolymer containing two or more types of 3-hydroxyalkanoic acids as monomer units. Examples of the copolymer include a copolymer of 3-hydroxybutyrate (3HB) and another 3-hydroxyalkanoic acid and a copolymer of 3-hydroxyalkanoic acids that contains at least 3-hydroxyhexanoate (3HH) as a monomer unit. Specifically, the following examples of the PHA are preferred because they are easy to produce industrially: poly(3-hydroxybutyrate) (PHB); poly(3-hydroxybutyrate-co-3-hydroxyvalerate) (PHBH); poly(3-hydroxybutyrate-co-3-hydroxyvalerate-co-3-hydroxyhexanoate); poly(3-hydroxybutyrate-co-3-hydroxyhexanoate); poly(3-hydroxybutyrate-co-4-hydroxybutyrate); poly(3-hydroxybutyrate-co-3-hydroxyoctanoate); and poly(3-hydroxybutyrate-co-3-hydroxyoctadecanoate).

The microorganisms that produce PHAs may be any microorganism having the ability to produce PHAs. For example, microorganisms isolated from nature, microorganisms deposited in strain depositories (IFO, ATCC, etc.), and genetically engineered microorganisms such as mutants and transformants of these microorganisms may be used.

Specific examples of the microorganisms include the following: Cupriavidus such as Cupriavidus necator; Alcaligenes such as Alcaligenes latus; Pseudomonas such as Pseudomonas putida, Pseudomonas fluorescens, Pseudomonas aeruginosa; Pseudomonas resinovorans, and Pseudomonas oleovorans; Bacillus such as Bacillus megaterium; Azotobacter; Nocardia; Aeromonas such as Aeromonas caviae and Aeromonas hydrophila; Ralstonia; Wautersia; and Comamonas (Microbiological Reviews, 54(4), 450-472 (1990)).

In addition to the above microorganisms, biological cells may also be used which have been modified to artificially produce PHAs by incorporating, e.g., PHA synthase genes using a genetic engineering technique. For example, the modified biological cells that artificially produce PHAs can be obtained by appropriately using, e.g., not only the microorganisms of the genera such as Cupriavidus; Alcaligenes Pseudomonas, Bacillus, Azotobacter; Nocardia, Aeromonas Ralstonia Wautersia, and Comamonas, as described above, but also gram-negative bacteria such as Escherichia, gram-positive bacteria such as Bacillus, and yeasts such as Saccharomyces, Yarrowia, and Candida.

PHBH of PHAs may be produced by using, e.g., microorganisms that inherently produce PHBH such as Aeromonas caviae and Aeromonas hydrophila, or biological cells that have been modified to artificially produce PHBH by introducing, e.g., PHA synthase genes into microorganisms that do not inherently produce PHBH using a genetic engineering technique. The host microorganisms into which genes are to be introduced may include, e.g., Cupriavidus necator. The PHA synthase genes may include, e.g., PHA synthase genes derived from Aeromonas caviae, Aeromonas hydrophila, or Chromobacterium sp. and variants of them. The variants may include, e.g., a base sequence encoding a PHA synthase in which an amino add group is deleted, added, inserted, or substituted.

The microorganisms may be cultured in the same manner as the conventional culture method for microorganisms except that the culture solution contains the lipid particles that are prepared by the above production method; specifically, the two-fluid nozzle is attached to a culture tank with the liquid supply port and the gas supply port located in the culture solution at a position lower than the liquid level of the culture solution in the culture tank, and fine lipid particles are prepared in the culture solution and supplied as a carbon source.

Any known method can be used to recover PHAs from the microorganisms that have been cultured by the above culture method. For example, the following method may be used. Upon completion of the culture, bacterial cells are separated from the culture solution with, e.g., a centrifuge. The bacterial cells are cleaned with distilled water, methanol, etc. and dried. Then, PHAs are extracted from the dried bacterial cells using an organic solvent such as chloroform. The solution containing the PHAs may be filtered to remove a bacterial cell component. Subsequently, a poor solvent such as methanol or hexane is added to the filtrate so that the PHAs are precipitated out. Further, the supernatant is removed by filtration or centrifugation and the precipitate is dried, thus recovering the PHAs.

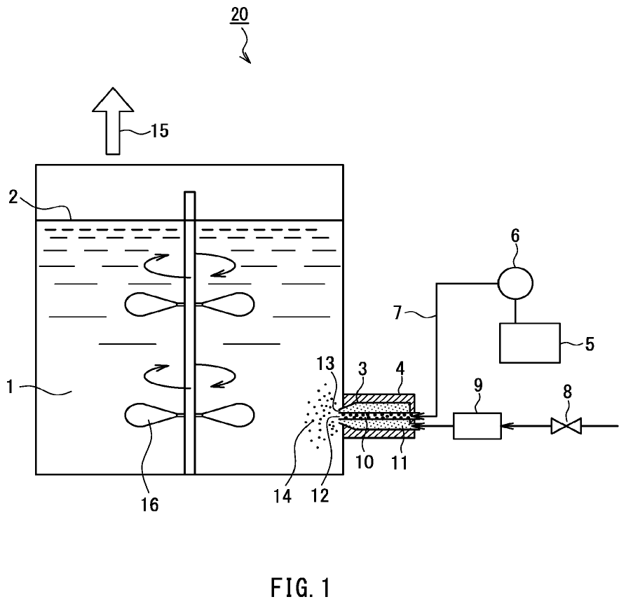

FIG. 1 is a schematic diagram of a device for producing lipid particles used in one or more embodiments of the present invention. The production device used in the production method of the present invention is not limited to that illustrated in FIG. 1.

As illustrated in FIG. 1, the production device 20 includes a container 1 (e.g., a culture tank) and a two-fluid nozzle 3. The two-fluid nozzle 3 is attached to a wall surface of the container 1 so that a liquid supply port 12 and a gas supply port 13 of the two-fluid nozzle 3 are located in the liquid at a position lower than the liquid level 2. The temperature of the two-fluid nozzle 3 can be adjusted by a heater 4. Moten lipids 10 are transferred to a liquid flow path of the two-fluid nozzle 3 from a tank 5 with a temperature control function through a line 7 with a temperature control function by using a tube pump 6. The molten lipids 10 are then supplied (injected) directly into the liquid from the liquid supply port (liquid injection hole) 12. A gas 11 is transferred to a gas flow path of the two-fluid nozzle 3 through a flow control valve 8 and a temperature regulating heater 9. The gas 11 is then supplied (injected) directly into the liquid from the gas supply port (gas injection hole) 13. The two-fluid nozzle 3 supplies the molten lipids 10 directly into the liquid at a temperature lower than the melting point of the lipids through the liquid supply port 12 while injecting the gas 11 directly into the liquid through the gas supply port 13. Thus, the injected molten lipids 10 are dispersed and atomized into particles in the liquid due to the injected gas 11 and the particles are solidified. Consequently, lipid particles 14 can be obtained. The ratio D50/Nd of the volume median diameter D50 of the target lipid particles to the orifice diameter Nd of the liquid supply port of the two-fluid nozzle is adjusted to be 0.0017 or more and 0.17 or less. The production device 20 also includes a stirrer 16 with an impeller. Thus, the lipid particles 14 can be uniformly dispersed in the culture solution. Reference numeral 15 is an exhaust line. The two-fluid nozzle 3 has a structure in which the liquid flow path is surrounded by the gas flow path. The gas functions as a heat insulator that prevents the molten lipids and the liquid flow path from being cooled by the culture solution. In a preferred embodiment where the temperature of the gas is higher than the melting point of the lipids, in particular, the function of the heat insulator will be further enhanced.

### EXAMPLES

Hereinafter, the present invention will be described in more detail by way of examples. However, the present invention is not limited to the following examples. Unless otherwise noted, the operation was performed at a temperature of 23° C. and a relative humidity of 60% in the following.

### Example 1

Lipid particles were produced by the production device of FIG. 1. First, 150 L of water (25° C.) was placed in a 200 L container. The two-fluid nozzle was mounted so that the outlet of the nozzle was located on the wall surface of the container and 300 mm above the lower end, and the liquid supply port and the gas supply port were arranged horizontally in the liquid. The two-fluid nozzle was an external mix two-fluid spray nozzle that has a circular spray pattern and is made of stainless steel (manufactured by Spraying Systems Co., 1/4JAUCO, orifice diameter of liquid feed portion (liquid supply port): 0.6 mm, torus area of gas feed portion: 1.6×10⁻⁶ m²). The two-fluid nozzle was preheated to 75 to 80° C. The air was heated to 60° C. before being transferred to the two-fluid nozzle and was adjusted so that the air flow rate was 10 L/min linear velocity: 104.2 m/sec). The temperature in the container was set to 34° C. A fatty acid distillate (PFAD with a melting point of 50° C., a density of 0.87 g/mL, and a solubility of 10 g/L or less at 25° C.) was heated to 60° C. The molten PFAD was added to the container at a rate (flow rate) of 0.2 to 0.86 mL/min and solidified in water. The ratio Vg/Vf of the injection linear velocity of the air (Vg) to the injection linear velocity of the molten fatty acid distillate (Vf) was 71. After a long-term operation for 6 hours, nozzle clogging did not occur and lipid particles were produced. In Example 1, the particle size distribution of the PFAD particles thus obtained was measured with a particle size distribution measuring device (MT3300EX II manufactured by Micotrac Inc.). As a result, the volume median diameter (D50) was 20.1 μm, the span [(D90−D10)/D50] was 2.51, and the ratio of the volume median diameter of the PFAD particles to the orifice diameter of the liquid supply port was 0.033.

### Comparative Example 1

The operation was performed in the same manner and under the same conditions as Example 1 except that the nozzle was not heated. Consequently, the nozzle was blocked within 5 minutes from the start of the supply (spraying) of the molten fatty acid distillate and the air, so that the device could not be operated.

### Examples 2 to 5

Fatty acid particles were produced in the same manner and under the same conditions as Example 1 except the following: 1 L of water (34° C.) was placed in a 5 L jar fermenter (BMS-5 L manufactured by Biott Corporation); the two-fluid nozzle was mounted so that the outlet of the nozzle was located on the wall surface of the fermenter and 50 mm above the lower end; the air transferred to the two-fluid nozzle was adjusted so that the air flow rate was 6 L/min (linear velocity: 62.5 m/sec); 5 g of PFAD was added to the fermenter at a rate of 1 mL/min (linear velocity: 0.06 m/sec); and the nozzle temperature and the PFAD temperature were set as shown in Table 1. In Examples 2 to 5, the ratio Vg/Vf of the injection linear velocity of the air (Vg) to the injection linear velocity of the molten fatty acid distillate (Vf) was 1060. The particle size distribution of the PFAD particles thus obtained was measured with a particle size distribution measuring device (MT3300EX II manufactured by Microtrac Inc.). Table 1 shows the results.

TABLE 1

| | Nozzle | PFAD | | | D50 of PFAD particles/ |
|---|---|---|---|---|---|
| | temperature | temperature | PFAD particles | | orifice diameter of |
| | (° C.) | (° C.) | D50(μm) | (D90-D10)/D50 | liquid supply port |
| Ex. 2 | 70 | 63 | 36.7 | 1.54 | 0.061 |
| Ex. 3 | 90 | 76 | 31.1 | 1.91 | 0.052 |
| Ex. 4 | 100 | 94 | 29.0 | 1.75 | 0.048 |
| Ex. 5 | 140 | 130 | 21.1 | 1.88 | 0.035 |

### Examples 6 to 13

Fatty acid particles were produced in the same manner and under the same conditions as Example 1 except the following: 3.5 L of water was placed in a 10 L jar fermenter (BMS-10L manufactured by Biott Corporation); 52.5 g of a fatty acid distillate (PFAD with a melting point of 50° C., a density of 0.87 g/mL, and a solubility of 10 g/L or less at 25° C.) was added to the fermenter; the air transferred to the two-fluid nozzle was adjusted so that the air flow rate was 10 L/min (linear velocity: 104.2 m/sec); and the rate of the PFAD added (PFAD flow rate) was set as shown in Table 2. The particle size distribution of the PFAD particles thus obtained was measured with a particle size distribution measuring device (MT3300EX II manufactured by Microtrac Inc.). Table 2 shows the results.

TABLE 2

| | | PFAD | | | | |
|---|---|---|---|---|---|---|
| | PFAD | linear | Gas linear | PFAD particles | | D50 of PFAD particles/ |
| | flow rate | velocity | velocity/PFAD | D50 | (D90 − D10)/ | orifice diameter of |
| | (mL/min) | (m/sec) | linear velocity | (μm) | D50 | liquid supply port |
| Ex. 6 | 2.2 | 0.13 | 792 | 19.2 | 1.09 | 0.032 |
| Ex. 7 | 3.9 | 0.23 | 452 | 19.2 | 1.15 | 0.032 |
| Ex. 8 | 5.1 | 0.30 | 346 | 23.9 | 1.02 | 0.040 |
| Ex. 9 | 5.5 | 0.32 | 323 | 24.1 | 0.97 | 0.040 |
| Ex. 10 | 6.2 | 0.37 | 285 | 24.8 | 0.96 | 0.041 |
| Ex. 11 | 14.7 | 0.87 | 120 | 27.9 | 0.94 | 0.046 |
| Ex. 12 | 20.2 | 1.19 | 87 | 28.0 | 1.05 | 0.047 |
| Ex. 13 | 90.4 | 5.33 | 20 | 33.6 | 1.41 | 0.056 |
| Ex/14 | 20.2 | 1.19 | 52 | 31.5 | 1.29 | 0.053 |

### Example 14

Fatty acid particles were produced in the same manner and under the same conditions as Example 1 except the following: 3.5 L of water (34° C.) was placed in a 10 L jar fermenter (BMS-10L manufactured by Biott Corporation); 52.5 g of a fatty acid distillate (PFAD with a melting point of 50° C., a density of 0.87 g/mL, and a solubility of 10 g/L or less at 25° C.) was added to the fermenter; the air transferred to the two-fluid nozzle was adjusted so that the air flow rate was 6 L/min (linear velocity: 62.5 m/sec); and the rate of the PFAD added was 20.2 mL/min (linear velocity: 1.19 m/sec). The ratio Vg/Vf of the injection linear velocity of the air (Vg) to the injection linear velocity of the molten fatty acid distillate (Vf) was 52. The particle size distribution of the PFAD particles thus obtained was measured with a particle size distribution measuring device (MT3300EX II manufactured by Microtrac Inc.). As a result, the volume median diameter (D50) was 31.5 μm, the span [(D90−D10)/D50] was 1.29, and the ratio of the volume median diameter of the PFAD particles to the orifice diameter of the liquid supply port was 0.053.

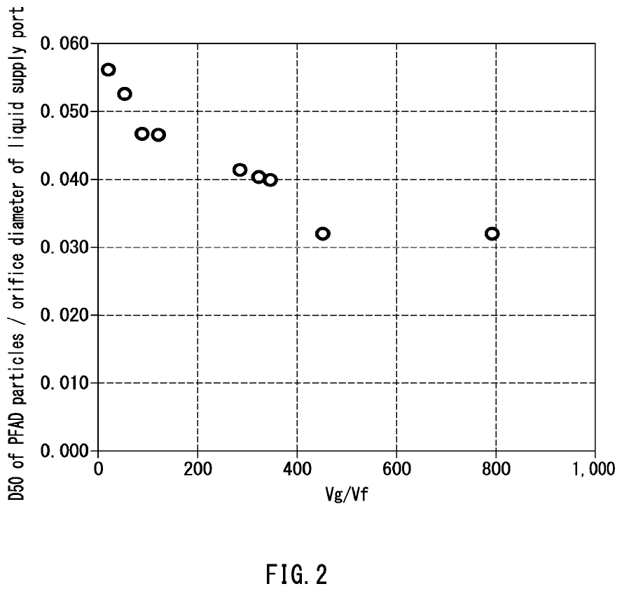

FIG. 2 shows a graph that plots the data of Vg/Vf versus the ratio of the volume median diameter of the PFAD particles to the orifice diameter of the liquid supply port in Examples 6 to 14. It is evident from FIG. 2 that there is a correlation between Vg/Vf and the ratio of the volume median diameter of the PFAD particles to the orifice diameter of the liquid supply port in the spraying of PFAD into the liquid with the two-fluid nozzle.

### Comparative Example 2

The PFAD was sprayed in the same manner and under the same conditions as Example 14 except the following: 1 L of water (34°) was placed in a cylindrical container with a diameter of 10 cm; the outlet of the external mix two-fluid spray nozzle was located at a height of 5 cm from the surface of the water; and the PFAD was ejected from the nozzle toward the surface of the water. The PFAD coagulated on the water surface and aggregated, resulting in coarse particles with a diameter of 3 mm or more.

The above results confirm that when the molten lipids are dispersed in a liquid with the external mix two-fluid spray nozzle, it is required that the molten lipids be sprayed into the liquid and the heated nozzle be used.

### Example 15

(Culture of Bacterial Cells Using the Spraying of Lipid into a Solution with an External Mix Two-Fluid Spray Nozzle)

A KNK-631 strain (see JP 2013-009627 A and WO 2016/114128 A1) was used for culture and production. The KNK-631 strain was subjected to seed culture, pre-culture, and main culture, and then bacterial cells were collected.

The composition of a seed culture medium included 1 w/v % Meat-extract, 1 w/v % Bacto-tryptone, 0.2 w/v % Yeast-extract, 0.9 w/v % Na₂HPO₄.12H₂O, and 0.15 w/v % KH₂O₄. The pH was adjusted to 6.8.

The composition of a pre-culture medium included 1.1 w/v % Na₂HPO₄.12H₂O, 0.19 w/v % KH₂PO₄, 1.29 w/v % (NH₄)₂SO₄, 0.1 w/v % MgSO₄.7H₂O, and 0.5 v/v % trace metal salt solution (in which 1.6 w/v % FeCl₃.6H₂O, 1 w/v % CaCl₂.2H₂O, 0.02 w/v % CoCl₂.6H₂O, 0.016 w/v % CuSO₄.5H₂O, and 0.012 w/v % NiCl₂.6H₂O were dissolved in 0.1N hydrochloric acid). Palm oil at a concentration of 10 g/L was added at once as a carbon source.

The composition of a main culture medium included 0.385 w/v % Na₂HPO₄.12H₂O, 0.067 w/v % KH₂PO₄, 0.291 w/v % (NH₄)₂SO₄, 0.1 w/v % MgSO₄.7H₂O, 0.5 w/v % trace metal salt solution (in which 1.6 w/v % FeCl₃.6H₂O, 1 w/v % CaCl₂.2H₂O, 0.02 w/v % CoCl₂.6H₂O, 0.016 w/v % CuSO₄.5H₂O, and 0.012 w/v % NiCl₂.6H₂O were dissolved in 0.1N hydrochloric acid), and 0.05 w/v % BIOSPUREX 200K (antifoaming agent, manufactured by Cognis Japan Ltd).

First, the seed culture was performed in such away that a glycerol stock (50 μL) of the KNK-631 strain was inoculated into the seed culture medium (10 mL) and cultured at 30° C. for 24 hours.

Then, the resulting seed culture solution was inoculated at 1.0 v/v % into 1.8 L of the pre-culture medium contained in a 3 L jar fermenter (MDL-300 manufactured by Marubishi Bioengineering Co., Ltd.). The operating conditions were as follows: culture temperature 30° C., stirring speed 600 rpm, and air flow rate 1.8 L/min. The pre-culture was performed for 24 hours with the pH being adjusted to 6.5. A 14% ammonium hydroxide aqueous solution was used for the pH control.

Next, the resulting pre-culture solution was inoculated at 1.0 v/v % into 6 L of the production culture medium contained in a 10 L jar fermenter (BMS-10L manufactured by Biott Corporation). The operating conditions were as follows: culture temperature 34° C., stirring speed 600 rpm, and air flow rate 6.0 L/min. The pH was adjusted to 6.5. A 14% ammonium hydroxide aqueous solution was used for the pH control. A PFAD was available from the FELDA and delivered via SUS (stainless steel) piping so as to avoid contact with iron piping. The PFAD (with a melting point of 50° C., a density of 0.87 g/mL, and a solubility of 10 g/L or less at 25° C.) was heated to 60° C. Using the external mix two-fluid spray nozzle that was preheated to 70° C., the molten PFAD was sprayed into the liquid and fed while the concentration in the culture solution was controlled. The two-fluid spray nozzle was the same as that used in Example 1. The air was heated to 60° C. before being transferred to the two-fluid nozzle and was adjusted so that the air flow rate was 6 L/min (linear velocity: 62.5 m/sec). The PFAD was added at a rate of 0.2 to 0.86 mL/min. A phosphoric acid solution was fed at a constant rate in the middle of the culture. The ratio Vg/Vf of the injection linear velocity of the air (Vg) to the injection linear velocity of the molten fatty acid distillate (Vf) was 1250. The main culture was performed for 48 hours. Upon completion of the culture, bacterial cells were collected by centrifugation. The bacterial cells were cleaned with methanol and lyophilized. The weight of the dried bacterial cells was measured. Then, 100 mL of chloroform was added to 1 g of the dried bacterial cells, and the mixture was stirred at room temperature for 24 hours. Thus, PHBH was extracted from the bacterial cells. The remainder after removal of the bacterial cells was concentrated in an evaporator to a total volume of 30 mL. Subsequently, 90 mL of hexane was gradually added to the concentrate, and the mixture was allowed to stand for 1 hour with gentle stirring. The precipitated PHBH was vacuum dried at 50° C. for 3 hours, so that PHBH was obtained. Table 3 shows the PHBH productivity and the carbon source yield. The PHBH productivity indicates the PHBH yield (g/L) per volume of the culture solution and the carbon source yield indicates the PHBH yield (g/g) per supply weight of the carbon source.

### Comparative Example 3

The culture was performed in the same manner and under the same conditions as Example 15 except that the PFAD spray was directed from a gas phase portion to the liquid. The sprayed PFAD coagulated on the water surface and aggregated, causing adhesion to the electrodes, stirring blades, and baffles in the jar fermenter. This made it difficult to culture cells.

TABLE 3

| | PHBH productivity | Carbon source yield |
|---|---|---|
| | (g/L) | (g/g) |
| Ex. 15 | 217 | 0.96 |
| Comp. Ex. 3 | — | — |

The above results confirm that even if lipids have a higher melting point than the culture temperature, the lipids can be efficiently and finely dispersed and atomized into particles in a culture solution at a temperature lower than the melting point of the lipids by spraying the molten lipids into the culture solution with the external mix two-fluid spray nozzle. Therefore, the lipids can be satisfactorily assimilated by microorganisms, and microbial metabolites can be efficiently produced.

The present invention includes, e.g., the following one or more embodiments.

[1] A method for producing lipid particles, in which molten lipids are solidified to form particles in a liquid, the lipids having a water solubility of 10 g/L or less at 25° C. and being solid at 25° C.,

the method comprising:

injecting the molten lipids directly into a liquid at a temperature lower than a melting point of the lipids through a liquid supply port of a two-fluid nozzle while injecting a gas directly into the liquid through a gas supply port of the two-fluid nozzle, so that the molten lipids are dispersed and atomized into particles in the liquid due to the gas and the particles are solidified to form lipid particles,

wherein the two-fluid nozzle is heated to a temperature at least 10° C. higher than the melting point of the lipids, and

a ratio D50/Nd of a volume median diameter D50 of the lipid particles to an orifice diameter Nd of the liquid supply port of the two-fluid nozzle is 0.0017 or more and 0.17 or less.

[2] The method according to [1], wherein a ratio Vg/Vf of an injection linear velocity of the gas Vg to an injection linear velocity of the molten lipids Vf is 10 or more and 2000 or less.

[3] The method according to [1] or [2], wherein the volume median diameter D50 of the lipid particles is 1 μm or more and 150 μm or less.

[4] The method according to any one of [1] to [3], wherein a span in a particle size distribution of the lipid particles represented by the following formula (1) is 0.5 or more and 3.0 or less:

Span=(D90−D10)/D50 (1).

[5] The method according to any one of [1] to [4], wherein the melting point of the lipids is 35° C. or more.

[6] The method according to any one of [1] to [5], wherein the lipids are derived from palm oil.

[7] The method according to any one of [1] to [6], wherein a temperature of the gas is equal to or higher than the melting point of the lipids and 200° C. or less.

[8] The method according to any one of [1] to [7], wherein the gas is air.

[9] The method according to any one of [1] to [8], wherein a temperature of the molten lipids is at least 5° C. higher than the melting point of the lipids.

[10] The method according to any one of [1] to [9], wherein the molten lipids are solidified to form particles in a microbial culture solution.

[11] The method according to [10], wherein the microbial culture solution is a culture solution for bacterial cells that produce polyhydroxyalkanoates.

[12] A method for culturing microorganisms, comprising:

preparing lipid particles in a culture solution by the method according to any one of [1] to [11], and

culturing microorganisms in the culture solution containing the lipid particles.

### DESCRIPTION OF REFERENCE NUMERALS

1 Container (culture tank)
2 Liquid level (liquid level of culture solution)
3 Two-fluid nozzle
4 Heater
5 Tank with temperature control function
6 Tube pump
7 Line with temperature control function
8 Flow control valve
9 Temperature regulating heater
10 Molten lipids
11 Gas
12 Liquid supply port (liquid injection hole)
13 Gas supply port (gas injection hole)
14 Lipid particles
15 Exhaust line
16 Stirrer with impeller
20 Production device for lipid particles

1: A method for producing lipid particles, comprising:
injecting molten lipids directly into a liquid at a temperature lower than a melting point of the lipids through a liquid supply port of a two-fluid nozzle while injecting a gas directly into the liquid through a gas supply port of the two-fluid nozzle, such that the molten lipids are dispersed and atomized into particles in the liquid due to the gas and that the particles are solidified to form the lipid particles,
wherein the lipids have a water solubility of 10 g/L or less at 25° C. and are solid at 25° C.,
the two-fluid nozzle is heated to a temperature at least 10° C. higher than the melting point of the lipids, and
a ratio D50/Nd of a volume median diameter D50 of the lipid particles to an orifice diameter Nd of the liquid supply port of the two-fluid nozzle is 0.0017 or more and 0.17 or less.

2: The method according to claim 1, wherein a ratio Vg/Vf of an injection linear velocity of the gas Vg to an injection linear velocity of the molten lipids Vf is 10 or more and 2000 or less.

3: The method according to claim 1, wherein the volume median diameter D50 of the lipid particles is 1 μm or more and 150 μm or less.

4: The method according to claim 1, wherein a span in a particle size distribution of the lipid particles represented by the following formula (1) is 0.5 or more and 3.0 or less:
Span=(D90−D10)/D50 (1).

5: The method according to claim 1, wherein the melting point of the lipids is 35° C. or more.

6: The method according to claim 1, wherein the lipids are derived from palm oil.

7: The method according to claim 1, wherein a temperature of the gas is equal to or higher than the melting point of the lipids and 200° C. or less.

8: The method according to claim 1, wherein the gas is air.

9: The method according to claim 1, wherein a temperature of the molten lipids is at least 5° C. higher than the melting point of the lipids.

10: The method according to claim 1, wherein the molten lipids are solidified to form the liquid particles in a microbial culture solution.

11: The method according to claim 10, wherein the microbial culture solution is a culture solution for bacterial cells that are capable of producing polyhydroxyalkanoates.

12: A method for culturing microorganisms, comprising:
preparing lipid particles in a culture solution by the method according to claim 1, and
culturing microorganisms in the culture solution containing the lipid particles.

13: The method according to claim 1, wherein the temperature of the liquid in which the molten lipids are solidified is 34 to 37° C.

14: The method according to claim 1, wherein the lipids comprise palm fatty acid distillate.

15: The method according to claim 12, wherein a temperature of the culture solution is 34 to 37° C.

16: The method according to claim 12, wherein the lipids comprise palm fatty acid distillate.
